(19) United States
(12) Patent Application Publication
(10) Pub. No.: US 2023/0294984 A1
(43) Pub. Date: Sep. 21, 2023

(54) Methane Reformer for the Production of Hydrogen and a Hydrocarbon Fuel

(71) Applicant: Syzygy Plasmonics Inc., Houston, TX (US)

(72) Inventors: Suman Khatiwada, Houston, TX (US); Trevor William Best, Houston, TX (US); Shreya Shah, Houston, TX (US); Syed Ali Gardezi, Houston, TX (US)

(73) Assignee: Syzygy Plasmonics Inc., Houston, TX (US)

(21) Appl. No.: 18/017,054

(22) PCT Filed: Jul. 20, 2021
(86) PCT No.: PCT/US2021/042448

§ 371 Date: Jan. 19, 2023

Related U.S. Application Data
(60) Provisional application No. 63/054,163, filed on Jul. 20, 2020.

(51) Int. Cl. C01B 3/34

(52) U.S. Cl. CPC C01B 3/346; C01B 2203/0233; C01B 2203/0283; C01B 2203/0475; C01B 2203/061; C01B 2203/1241

(57) ABSTRACT

The present disclosure is directed to systems and methods for reforming methane into hydrogen and a hydrocarbon fuel. In example embodiments, the methane reformer integrates a photocatalytic steam methane reforming (P-SMR) system with a subsequent photocatalytic dry methane reforming (P-DMR) system.

### CROSS-REFERENCE TO RELATED APPLICATIONS

The present application claims priority to and hereby incorporates by reference the entirety of U.S. Provisional Pat. Application No. 63/054,163, filed Jul. 20, 2020.

### BACKGROUND OF DISCLOSURE

### Field of Disclosure

The present disclosure is directed to systems and methods for reforming methane into hydrogen and a hydrocarbon fuel. In example embodiments, the methane reformer integrates a photocatalytic steam methane reforming (P-SMR) system with a subsequent photocatalytic dry methane reforming (P-DMR) system.

### Technical Background

Conventional Steam Methane Reforming (SMR) systems, such as the one illustrated in FIG. 1, can be used to produce syngas (hydrogen and carbon monoxide) from, for example, methane (natural gas), according to the following equilibrium:

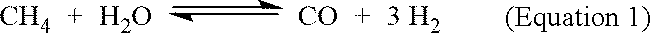

The conventional SMR has several disadvantages. For example, SMR is sensitive to sulfur that may be present in the pipeline quality gas and requires desulfurization (i.e., a combination of hydrodesulfurization (HDS) catalyst and ZnO adsorbent bed). In addition, conventional SMR is a heat intensive endothermic reactor, and hydrogen production is limited due to conversion limitation associated with near cracking temperatures. This limitation is overcome via a high and low temperature water gas shift reactor (WGS), installed in series. Further, high temperature operation of SMR produces significant quantities of green-house carbon monoxide (CO), which necessitates the installation of WGS reactors.

In addition, the conventional SMR generally has two carbon dioxide (CO2) exhaust streams, which require removal of CO2. The first CO2 exhaust stream results from natural gas and air being used as fuel to provide energy to the SMR reactor. This creates a “stack gas” stream that has dilute CO2 and other gases, such as nitrogen oxides (NOX) and sulfur oxides (SOX). The process to capture or utilize CO2 from the stack gas stream is complex and expensive. The second CO2 exhaust stream is produced as a part of the process gas, and contains concentrated CO2 that is easier to capture or utilize. The amount of CO2 released to the atmosphere from both of these streams makes conventional SMR a significant emitter of greenhouse gases. In plants that contain equipment to capture CO2 from these streams, the capital expenditure for such equipment becomes an appreciable portion of the overall plant cost.

One of the traditional methods employed for CO2 removal is a combination absorber-regenerator setup that employs hot potash or amine based liquid absorbents, such as monoethanolamine (MEA) or activated methyl diethanol amine (aMDEA). Not only does this system require a high pressure (close to 400 psi(g), for liquid entering the absorbers) and high temperature (close to 200° C. at regenerator reboiler), but amine-based liquids used in the system can be corrosive in nature. These limitations require high grade costly materials; i.e., the whole tower has to be made from stainless steel or require the injections of a passivation agent, such as vanadium pentaoxide (V₂O₅), and continuous iron monitoring. Foaming is another common issue. Excessive foaming can lead to carry over to the downstream system and have a negative effect. Finally, solution chemistry needs to be analyzed at regular frequency to maintain the necessary rate of absorption and address any system losses.

The conventional SMR design also necessitates a fully functional burner management system (BMS) to ensure the safe light-up and light-off of gas/liquid fuel operated burners. A BMS system has significant steps after which the permissive is issued to light up burners. This sequence conventionally includes purging of the furnace to get rid of the flammables from the firing (if any) by running blowers or ID fans near their top speeds. Once the purge sequence is completed, a tightness test ensures leak proofing of the fuel circuit, after which the pilot lights-up and then, based on the predetermined or operationally required sequence, the main burners light-up and the system is pressurized. As evident, it is a complicated system with excessive boot strapping. Further, any leakage in the fuel system renders the entire sequence useless. Additionally, the furnace ramp-up or ramp-down requires a lot of time and labor. A commercial reformer with close to hundred burners requires manual operation every time pressure is stepped up or lowered. A combination of block and regulating valves (i.e., control valves) ensures precise control and, if needed, fail-safe shutdown, but requires constant vigilance on the part of board and field operators.

Therefore, there remains a need for effective systems for methane reforming that do not have the drawbacks of the currently used conventional SMR systems.

### SUMMARY OF DISCLOSURE

One aspect of the disclosure provides a system for recovering syngas (i.e., hydrogen and carbon monoxide) from a methane feedstock. Such system includes:
a first stage comprising a photocatalytic steam methane reformer, the first stage configured to produce at least a carbon dioxide stream and a hydrogen stream from the methane feedstock; and
a second stage, adjacent to and downstream from the first stage, and comprising a photocatalytic dry methane reformer configured to produce the syngas from a second methane feedstock and the carbon dioxide stream produced in the first stage.

The system of the disclosure may be used in methods of preparing zero-emission hydrogen in addition to another low- or zero-emission product, such as methanol or dimethyl ether (DME). Thus, another aspect of the disclosure provides methods for transforming a methane feedstock into syngas. Such methods include:
providing the methane feedstock to a first stage comprising a photocatalytic steam methane reformer as described herein to obtain at least a carbon dioxide stream and a hydrogen stream; and
providing the carbon dioxide stream to a second stage comprising a photocatalytic dry methane reformer as described herein to produce the syngas.

Another aspect of the disclosure provides a method for preparing a hydrocarbon fuel, such as methanol or dimethyl ether, from a methane feedstock. Such method includes:
providing the methane feedstock to a first stage comprising a photocatalytic steam methane reformer as described herein to obtain at least a carbon dioxide stream and a hydrogen stream;
providing the carbon dioxide stream to a second stage comprising a photocatalytic dry methane reformer as described herein to produce the syngas; and
providing the syngas to a third stage comprising a reactor to obtain methanol or dimethyl ether.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the invention will become apparent to those skilled in the art from this detailed description.

### BRIEF DESCRIPTION OF THE DRAWINGS

The accompanying drawings are included to provide a further understanding of the systems and methods of the disclosure, and are incorporated in and constitute a part of this specification. The drawings illustrate one or more embodiment(s) of the disclosure and, together with the description, serve to explain the principles and operation of the disclosure.

FIG. 1 is a process flow diagram illustrating a conventional SMR system.

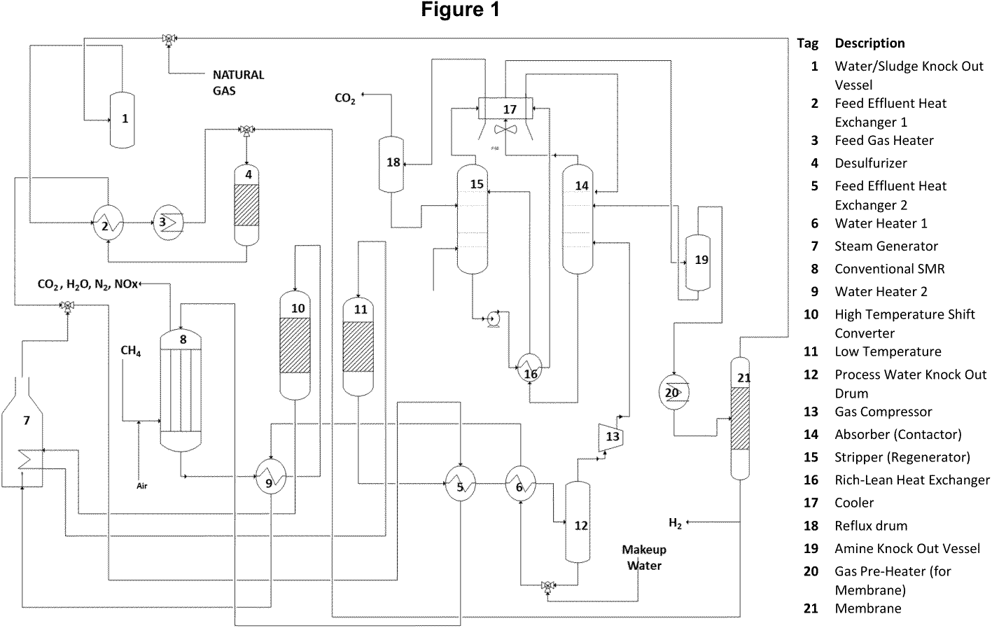

FIG. 2 is a process flow diagram illustrating a methane reformer system for producing syngas, according to a first example embodiment.

FIG. 3 is a process flow diagram illustrating a methane reformer system for producing syngas, according to a second example embodiment.

FIG. 4 is a schematic diagram illustrating a process for producing syngas, according to example embodiments.

FIG. 5 is a process flow diagram illustrating a methane reformer system for producing hydrogen and methanol, according to a third example embodiment.

FIG. 6 is a process flow diagram illustrating a methane reformer system having an Organic Rankin Cycle (ORC) unit for producing hydrogen and methanol, according to a fourth example embodiment.

### DETAILED DESCRIPTION

Example methods and systems are described herein. Any example embodiment or feature described herein is not necessarily to be construed as preferred or advantageous over other embodiments or features. The example embodiments described herein are not meant to be limiting. It will be readily understood that certain aspects of the disclosed systems and methods can be arranged and combined in a wide variety of different configurations, all of which are contemplated herein.

In view of the present disclosure, the systems and methods described herein can be configured by the person of ordinary skill in the art to meet the desired need. In general, the disclosed systems, methods, and apparatus provide improvements in photocatalysis systems and processes. Specifically, the invention provides an improved electrified SMR reactor, a photocatalytic steam methane reformer (P-SMR), that does not burn hydrocarbon fuel but instead uses electricity to make hydrogen and CO₂ (as a process by-product). This CO₂ is then utilized in a second electrified reactor, a photocatalytic dry methane reformer (P-DMR), to create synthetic gas (or syngas). This syngas can be sent to a synthesis reactor to produce a liquid fuel such as methanol or dimethyl ether. As a result, in certain embodiments, this system uses less natural gas than the traditional process, does not emit CO₂ to the environment, and is capable of using renewable electricity to operate. In certain embodiments, the system and method of the disclosure can be advantageously used to produce other commercially advantageous materials such as methanol or dimethyl ether. The system and methods of the disclosure, in certain embodiments, eliminate the capital cost and operational complexity associated with BMS and CO₂ capture equipment in conventional plants. In certain embodiment, waste heat generated at one part of the system (e.g., in a reactor cooling jacket) can be advantageously utilized elsewhere in the system to increase overall operational efficiency of the system.

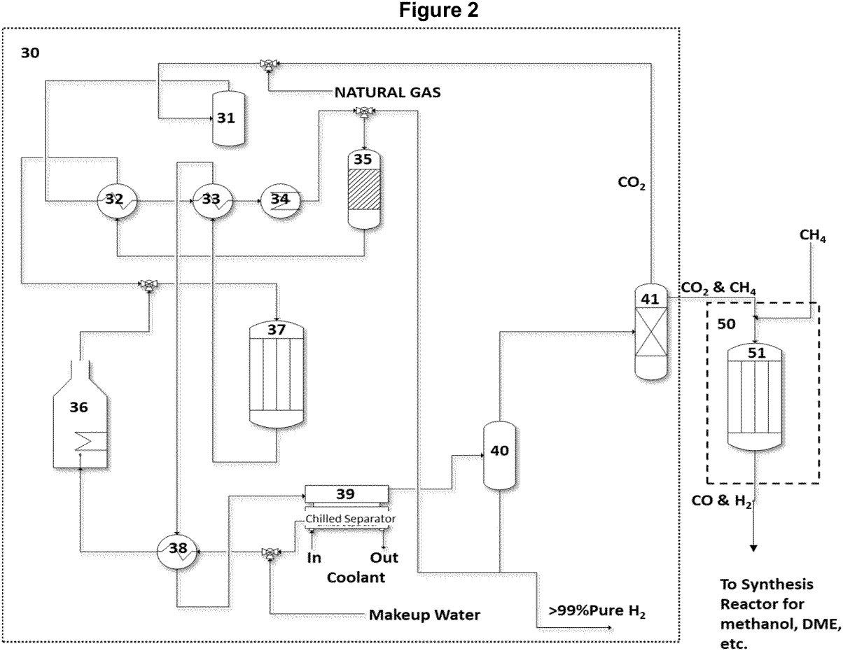

As provided above, the disclosure provides a system for recovering syngas (i.e., hydrogen and carbon monoxide) from a methane feedstock. Specifically, as illustrated in FIG. 2, the system of the disclosure includes a first stage (30) configured to produce at least a carbon dioxide stream and a hydrogen stream from the methane feedstock. The first stage comprises a photocatalytic steam methane reformer (P-SMR) (37). The P-SMR (37) is configured for contacting the methane feedstock with steam in the presence of a first plasmonic photocatalyst to form a first reaction product stream comprising hydrogen and carbon monoxide.

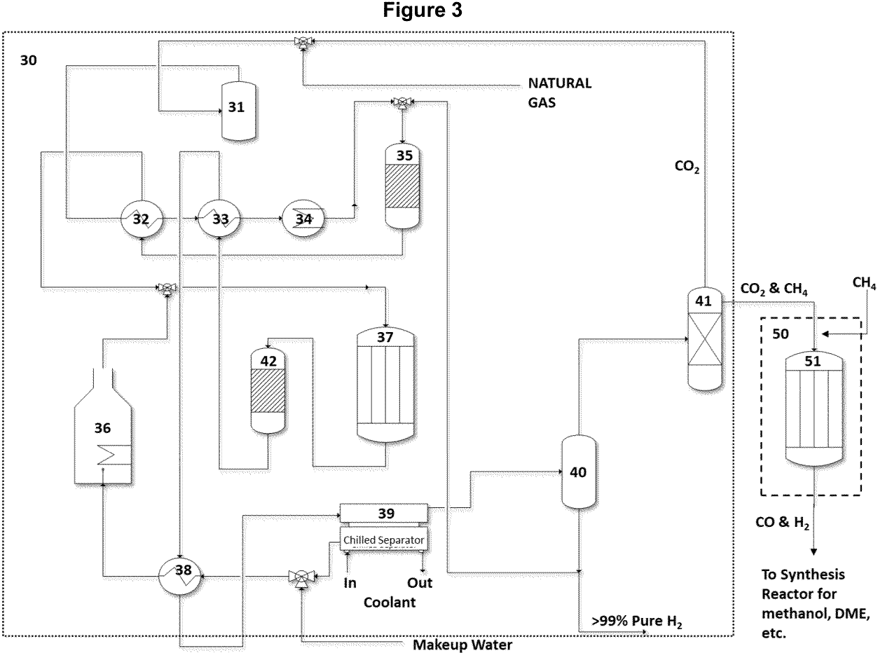

In certain embodiments, as illustrated in FIG. 3, the first stage (30) comprises the photocatalytic steam methane reformer (37) and a water-gas shift (WGS) reactor (42). The WSG reactor (42) is configured for contacting the first reaction product stream with water to form a water-gas shift stream comprising hydrogen and carbon dioxide.

In certain embodiments, the first stage (30) may further comprise a separation unit configured for separating carbon dioxide from the water-gas shift stream to obtain the carbon dioxide stream and the hydrogen stream. As illustrated in FIGS. 2 and 3, in certain embodiments, the separation unit may comprise pressure swing adsorption (PSA) hydrogen purification unit (40) and/or CO₂ absorption unit (41). Note that, while FIGS. 2 and 3 illustrate a feedback CO₂ stream from the CO₂ absorption unit (41), such a stream is optional and need not be utilized in some embodiments. Similarly, other illustrated components and streams may be omitted in some embodiments, depending on the particular application and/or system scale being implemented.

As illustrated in FIG. 2 and FIG. 3, in certain embodiments, the first stage (30) may optionally contain one or more of water/sludge knock out vessel (31), feed effluent H.X-1 and/or H.X.-2 (32 and/or 33), trim heater-cooler (e.g., electrical) (34), desulfurizer (35), steam generator (36), water heater (38), and chiller (39).

One of the drawbacks of the conventional process is the loss of heat from the SMR. The conventional SMR process is only approximately 50% efficient — half of the supplied power is lost as heat rejected through the walls of SMR. Further, in the conventional design, a significant amount of heat is lost in condensing the gas. The inventors have determined that heat can be recovered using the organic Rankine cycle (ORC). At appropriate scale, an ORC cycle can give as high as 40% exergy efficiency, thus making it an attractive option for increasing the energy efficiency of the process from 45% to as high as about 70%. Thus, in certain embodiments, the first system of the disclosure (30) may further comprise an organic Rankine cycle (ORC) configured to generate electricity within the system using process waste heat. In larger systems, the available heat is even higher in grade. Thus, in certain embodiments, the system may further comprise a steam turbine configured to generate power in-situ.

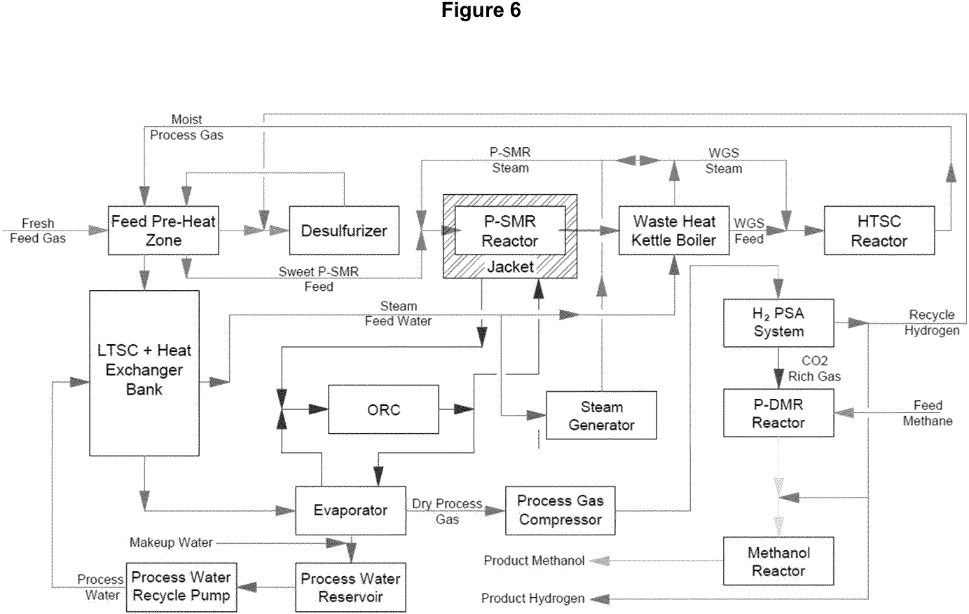

A more detailed illustration of an embodiment utilizing an ORC unit for in-situ power generation is shown in the process flow diagram of FIG. 6. The system of FIG. 6 produces hydrogen and methanol and utilizes an ORC unit for improved efficiency. As shown, the system includes the ORC unit and its evaporator in parallel with the P-SMR reactor. In particular, the ORC unit uses waste heat from a fluid cooling system (e.g., a cooling jacket or reservoir) associated with the P-SMR reactor to generate electricity. Such electricity may, in turn, be used to power ancillary electrical components associated with the system, such as control electronics, pumps, sensors, or other electrically powered components. This can reduce the required electricity input to be generated by other external means, such as conventional grid-generated power or renewable (e.g., solar or wind) power generated locally or remotely.

As mentioned, the fluid cooling system described above for in-situ power generation may be in the form of a cooling jacket or reservoir associated with the P-SMR reactor. For example, each individual reactor cell may be surrounded by a fluid jacket through which coolant (e.g., water) is moved. For example, coolant may be pumped or otherwise be moved through the cooling jacket to remove heat generated by a reactor cell surrounded by the cooling jacket. In the case of an annular-shaped reactor cell, the fluid cooling system may additionally or alternatively include an interior cooling jacket or reservoir in a center portion of the reactor cell, such that the interior cooling jacket itself is surrounded by the annular-shaped reactor cell. Other configurations of fluid cooling systems for use by the ORC are possible and intended to fall within the scope of the present disclosure. For example, a cooling system that removes heat from more than one reactor cell or that is associated with a multi-cell reactor (or a multi-reactor reformer) may additionally or alternatively supply waste heat for in-situ power generation by the ORC unit.

In certain other embodiments, no in-situ power is generated in the system of the disclosure. For example, the water gas shift reactor is exothermic in nature and the process heat integration helps in heating water for steam generation in a waste heat boiler. The main steam generator / waste heat boiler uses the hot SMR exit stream and cools the process gas to a high temperature shift converter (HTSC) inlet temperature. Shift conversion effectively reduces CO to trace amounts (less than 1 %, e.g., about 0.2%) by converting it to CO₂. The stream at the exit of the shift reactor is dried (to get rid of the excess water) and then compressed to approximately 10 Bar (145 psi(g)). In certain example embodiments, the P-SMR of the disclosure has a maximum inlet pressure of about 100 psi(g). In certain embodiments, the gas will be further pressurized (to approximately 10 bar (i.e., 145 psi(g)), before going to a unit to separate hydrogen.

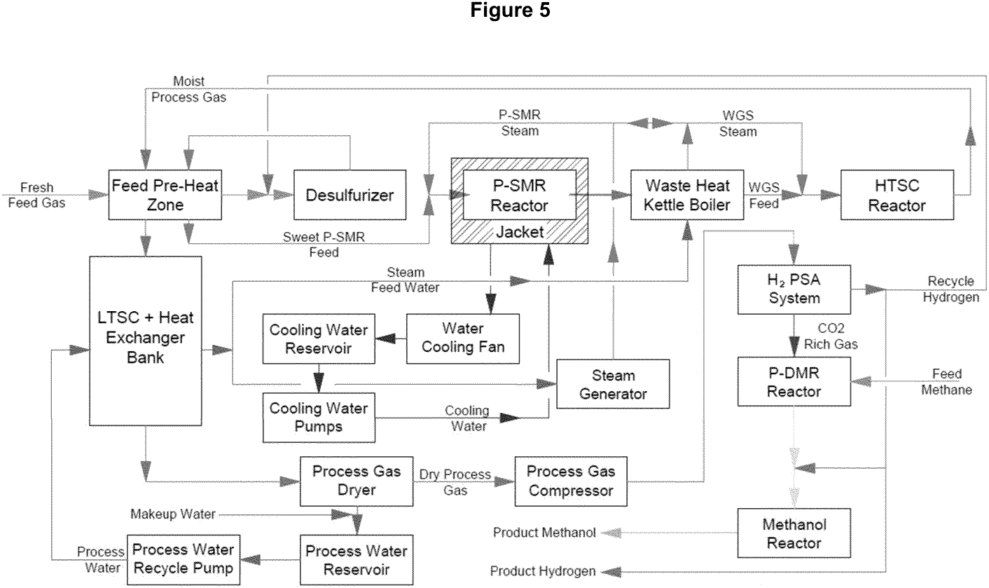

FIG. 5 is a is a process flow diagram illustrating a methane reformer system for producing hydrogen and methanol, according to a third example embodiment. The system of FIG. 5 is similar to that illustrated in FIG. 6, except the system of FIG. 5 does not generate in-situ power, but instead simply recirculates coolant (e.g., water) through the P-SMR reactor’s cooling jacket after removing generated heat. As shown, one or more cooling fans, reservoirs, and/or pumps may be used to recirculate coolant through the cooling jacket.

The system of the disclosure also includes a second stage (50), adjacent to and downstream from the first stage (30), and comprising a photocatalytic dry methane reformer (P-DMR) (51) configured to produce the syngas from a second methane feedstock and the carbon dioxide stream produced in the first stage (30).

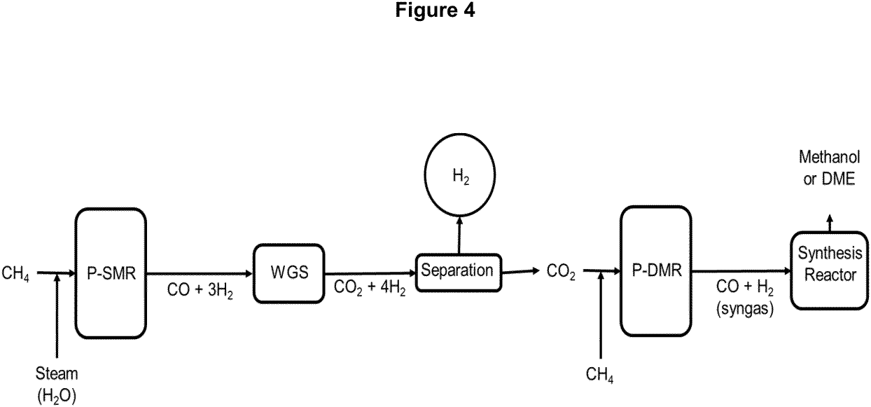

In certain embodiments, the system of the disclosure further includes a third stage, adjacent to and downstream from the second stage (50), and comprising a synthesis reactor configured to produce methanol or dimethyl ether from the syngas produced in the second stage, as illustrated in FIG. 4.

The example reactions carried out in the second and third stage to obtain, for example, methanol, are as follows:
Step 1 — Dry Methane Reforming (DMR):

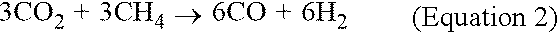

Step 2 — Water Gas Shift (WGS):

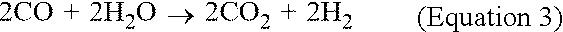

Sum of steps 1 & 2:

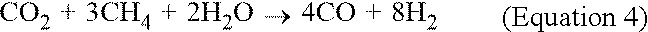

Step 3 — Methanol Synthesis:

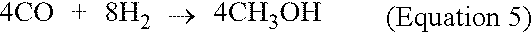

Sum of steps 1, 2 & 3:

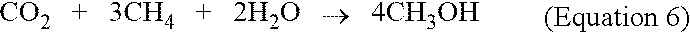

As described above in Equation 2 (Step 1), the output of the P-DMR reactor is syngas or synthetic gas that is a mixture of CO and H₂. Syngas is the starting feedstock for many hydrocarbon fuels such as methanol and dimethyl ether. The technologies to convert syngas to hydrocarbon fuels are mature and commercial and would be apparent to those of ordinary skill in the art.

The syngas from the second stage (50) generally contains carbon monoxide and hydrogen in about a 1:1 ratio. In certain embodiments, a hydrogen stream is provided to the synthesis rector in the third stage so that the ratio of carbon monoxide and hydrogen in the synthesis reactor is about 1:2 (for example, as shown in Equation 5). The hydrogen stream may be provided directly to the synthesis reactor, or it might be pre-mixed with the syngas stream prior to introduction to the synthesis reactor. In certain embodiments, the hydrogen stream introduced into the synthesis reactor is obtained in the first stage (30), such as from the PSA hydrogen purification unit (40).

In certain other embodiments, a shift reactor may be added in the second stage (50) adj acent to and downstream from the P-DMR (51) and configured to produce the hydrogen stream that is provided to the synthesis reactor. This process is illustrated by Equation 3 and Equation 4.

In certain other embodiments, the second stage (50) comprises a hydrogen separation membrane adjacent to and downstream from the P-DMR (51) and configured to produce the hydrogen stream that is provided to the synthesis reactor.

The choice of hydrogen separation technology directly depends on the end usage. Emerging gas separation technologies include membrane separation, which has the advantages of flexible and simple operation, compact structure, low energy consumption, and environmental friendliness. The performance of membrane materials is the most critical factor determining the H₂ separation and purification effects of the membrane. Commonly used membrane materials primarily include metal and polymer membranes, and novel membrane materials, such as nanomaterial membrane, CMSM, and MOF membranes may exhibit preferable separation performance. No single membrane type system can provide 99% + purity. Further, a membrane system is highly sensitive to condensation of water, as it forms a barrier on the surface of the membrane and slows down the permeation rate. While amine vapor has a negligible impact on the membrane, the possibility of foaming and carry-over requires additional unit operations, such as utilizing a heater and coalescing filter in conventional SMR systems. If liquid MEA / MDEA carries-over, then the only choice may be to shut-down the facility and replace the membrane.

In contrast to the conventional SMR systems, the system of the disclosure can utilize the hydrogen separation membrane without the concerns of the above-noted drawbacks. Thus, in certain embodiments, the hydrogen separation membrane used in the system of the disclosure is a pressure swing adsorption (PSA) hydrogen unit. The PSA separation effect primarily depends on the type of adsorbent and the technical process used. Because H₂ significantly differs from most gas molecules, such as CO₂, CO, and CH₄, in terms of static capacity, it is very suitable for PSA separation and purification. In certain examples, purities as high as 99% can be achieved.

As provided above, the system of the disclosure comprises the photocatalytic steam methane reformer (P-SMR). For example, such P-SMR may include:
a housing;
at least one reactor cell disposed within an interior of the housing, the at least one reactor cell comprising an enclosure and a first plasmonic photocatalyst on a first catalyst support disposed within the at least one enclosure, wherein the enclosure is optically transparent and comprises at least one input for the methane feedstock to enter the at least one cell and at least one output for the first reaction product stream to exit the at least one cell; and
at least one light source, wherein, upon application of the at least one light source, the reactor cell is configured to form the first reaction product stream from the methane feedstock.

Similarly, the system of the disclosure comprises the photocatalytic dry methane reformer (P-DMR). For example, such P-DMR may include:
a housing;
at least one reactor cell disposed within an interior of the housing, the at least one reactor cell comprising an enclosure and a second plasmonic photocatalyst on a second catalyst support disposed within the at least one enclosure, wherein the enclosure is optically transparent and comprises one or more inputs for the second methane feedstock and the carbon dioxide stream to enter the at least one cell and at least one output for the syngas to exit the at least one cell; and
at least one light source, wherein, upon application of the at least one light source, the reactor cell is configured to form the syngas from the second methane feedstock and the carbon dioxide stream.

Examples of other suitable P-SMR and P-DMR are described in International Patent Publication Nos. WO 2019/005777, WO 2019/005779, WO 2020/146799, WO 2020/146813, and WO 2018/231398, each incorporated by reference herein.

The reactor cells of the P-SMR and P-DMR of the disclosure require one or more plasmonic photocatalysts comprising a catalyst coupled to a plasmonic material, such as through a physical, electronic, thermal, or optical coupling. Without being bound by theory, the plasmonic material is believed to act as an optical antenna capable of absorbing light due to the unique interaction of light with plasmonic materials and, as a result, generates a strong electric field on and near the plasmonic material (i.e., as a result of collective oscillation of electrons within the plasmonic material). This strong electric field on and near the plasmonic material allows for coupling between the catalyst and the plasmonic material, even when the catalyst and the plasmonic material are separated by distances of up to about 20 nm or more.

In general, the plasmonic material may be any metal, metal alloy, metalloid element, or its alloy. In some embodiments, the plasmonic material of the disclosure is selected from gold, gold alloy, silver, silver alloy, copper, copper alloy, aluminum, or aluminum alloy. In the present disclosure, the term “alloys” is intended to cover any possible combination of metals. For example, the alloys may be binary alloys such as AuAg, AuPd, AuCu, AgPd, AgCu, etc., or they may be ternary alloys, or even quaternary alloys. In certain embodiments, the plasmonic material of the disclosure is aluminum, copper, silver, or gold.

In general, the catalyst material coupled to the plasmonic material may be any compound capable of catalyzing the required reaction (i.e., the first catalyst coupled to the plasmonic material may be any compound capable of catalyzing a SMR reaction (e.g., even if it were not coupled to a plasmonic material)). In some embodiments, the catalyst of the disclosure may be any metal or metalloid element, and any alloy, oxide, phosphide, nitride, or combination thereof of said elements. For example, the first catalyst and/or the second catalyst of the disclosure may independently comprise catalytically active iron, nickel, cobalt, platinum, palladium, rhodium, ruthenium or any combination thereof. The catalyst of the disclosure may comprise any alloy, oxide, phosphide, or nitride of catalytically active iron, nickel, cobalt, platinum, palladium, rhodium, or ruthenium. In some embodiments, the catalyst of the disclosure comprises catalytically active iron or nickel.

Examples of suitable plasmonic photocatalysts are provided in D. F. Swearer et al., “Heterometallic antenna-reactor complexes for photocatalysis,” Proc. Natl. Acad. Sci. U.S.A. 113, 8916-8920, 2016; Linan Zhou et al. “Quantifying hot carrier and thermal contributions in plasmonic photocatalysis,” Science, 69-72, 05 Oct. 2018; Linan Zhou et al., “Light-driven methane dry reforming with single atomic site antenna-reactor plasmonic photocatalysts,” Nature Energy, 5, 61-70, 2020, each incorporated by reference herein.

As provided above, the system of the disclosure may be used in methods of preparing zero-emission hydrogen in addition to another, low- or zero-emission product, such as methanol or dimethyl ether (DME).

Thus, another aspect of the disclosure provides methods for transforming a methane feedstock into syngas. Such methods include:
providing the methane feedstock to a first stage comprising a photocatalytic steam methane reformer as described herein to obtain at least a carbon dioxide stream and a hydrogen stream; and
providing the carbon dioxide stream to a second stage comprising a photocatalytic dry methane reformer as described herein to produce the syngas.

In such methods, for example, in the first stage, the methane feedstock is provided to the photocatalytic steam methane reformer to form a first reaction product stream comprising hydrogen and carbon monoxide; followed by providing the first reaction product stream and water to a water-gas shift reactor to form a water-gas shift stream comprising hydrogen and carbon dioxide. Specifically, in the photocatalytic steam methane reformer, the methane feedstock is distributed into a plurality of reactor cells disposed within a photocatalytic steam methane reformer housing, wherein each reactor cell comprises an optically transparent enclosure and a first plasmonic photocatalyst on a first catalyst support disposed within the optically transparent enclosure. This is followed by illuminating, via at least one light source, an interior of the photocatalytic steam methane reformer housing to cause the plurality of reactor cells to transform the methane feedstock into the first reaction product stream comprising hydrogen and carbon monoxide; and accumulating the first reaction product stream from the plurality of reactor cells.

In certain embodiments of the methods of the disclosure, the water-gas shift stream comprising hydrogen and carbon dioxide is provided to a separation unit to obtain the carbon dioxide stream and the hydrogen stream.

Finally, in the second stage, the methods of the disclosure comprise:
in the photocatalytic dry methane reformer, distributing the carbon dioxide stream and a second methane feedstock into a plurality of reactor cells disposed within a photocatalytic dry methane reformer housing, wherein each reactor cell comprises an optically transparent enclosure and a second plasmonic photocatalyst on a second catalyst support disposed within the optically transparent enclosure;
illuminating, via at least one light source, an interior of the photocatalytic dry methane reformer housing to cause the plurality of reactor cells to transform the carbon dioxide and methane into the syngas; and
accumulating the syngas from the plurality of reactor cells.

Another aspect of the disclosure includes methods for preparing methanol or dimethyl ether from a methane feedstock. In such methods, the syngas obtained in the second stage is provided to a third stage comprising a synthesis reactor to obtain methanol or dimethyl ether.

In certain embodiments, a hydrogen stream is provided to the synthesis rector in the third stage so that the ratio of carbon monoxide and hydrogen in the reactor is about 1:2.

Various example embodiments described herein can be used to provide one or more benefits, such as benefits relating to reduced-emission chemical production. In one example use case, methane from a dairy farm, landfill, or well-site flare gas can be used to make low / zero emission hydrogen from that methane, without significant carbon emissions into the atmosphere. By processing the P-SMR’s CO₂ waste stream in the immediately adjacent and downstream P-DMR reactor, the waste CO₂ and methane (both potent greenhouse gases) can be processed into another “green” product, such as methanol or DME, for example. In certain embodiments, the methods of the disclosure are lower cost, less complex, and an environmentally friendly replacement for traditional SMR plants in oil refineries, ammonia plants, and methanol plants. The systems and methods of the disclosure may be used, for example, as a source of hydrogen fuel for distributed and point-of-use production of hydrogen for fuel cell vehicle applications.

The above detailed description describes various features and functions of the disclosed systems, devices, and methods with reference to the accompanying figures. While various aspects and embodiments have been disclosed herein, other aspects and embodiments will be apparent. The various aspects and embodiments disclosed herein are for purposes of illustration only and are not intended to be limiting.

Some embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

What is claimed:

1. A system for recovering syngas from a methane feedstock, comprising:
a first stage comprising a photocatalytic steam methane reformer, the first stage configured to produce at least a carbon dioxide stream and a hydrogen stream from the methane feedstock; and
a second stage, adjacent to and downstream from the first stage, comprising a photocatalytic dry methane reformer configured to produce the syngas from a second methane feedstock and the carbon dioxide stream produced in the first stage.

2. The system of claim 1, wherein the first stage comprises:
the photocatalytic steam methane reformer configured for contacting the methane feedstock with steam in the presence of a first plasmonic photocatalyst to form a first reaction product stream comprising hydrogen and carbon monoxide; and
a water-gas shift reactor configured for contacting the first reaction product stream with water to form a water-gas shift stream comprising hydrogen and carbon dioxide.

3. The system of claim 2, wherein the first stage further comprises:
a separation unit configured for separating carbon dioxide from the water-gas shift stream to obtain the carbon dioxide stream and the hydrogen stream.

4. The system of claim 2, wherein the photocatalytic steam methane reformer comprises:
a housing;
at least one reactor cell disposed within an interior of the housing, the at least one reactor cell comprising an enclosure and the first plasmonic photocatalyst on a first catalyst support disposed within the enclosure, wherein the enclosure is optically transparent and comprises at least one input for the methane feedstock to enter the at least one reactor cell and at least one output for the first reaction product stream to exit the at least one reactor cell; and
at least one light source, wherein, upon application of the at least one light source, the reactor cell is configured to form the first reaction product stream from the methane feedstock.

5. The system of claim 2, wherein the first plasmonic photocatalyst comprises a first catalyst coupled to a plasmonic material.

6. The system of claim 5, wherein the first catalyst comprises catalytically active iron, nickel, cobalt, platinum, palladium, rhodium, or ruthenium, and wherein the plasmonic material is aluminum, copper, silver, or gold.

7. The system of claim 1, wherein the first stage comprises an organic Rankine cycle to generate electricity within the system using process waste heat.

8. The system of claim 1, wherein the photocatalytic dry methane reformer comprises:
a housing;
at least one reactor cell disposed within an interior of the housing, the at least one reactor cell comprising an enclosure and a plasmonic photocatalyst on a catalyst support disposed within the enclosure, wherein the enclosure is optically transparent and comprises one or more inputs for the second methane feedstock and the carbon dioxide stream to enter the at least one cell and at least one output for the syngas to exit the at least one cell; and
at least one light source, wherein, upon application of the at least one light source, the reactor cell is configured to form the syngas from the second methane feedstock and the carbon dioxide stream.

9. (canceled)

10. (canceled)

11. The system of claim 1, further comprising a third stage, adjacent to and downstream from the second stage, comprising a synthesis reactor configured to produce methanol or dimethyl ether from the syngas produced in the second stage.

12. The system of claim 11, wherein a hydrogen stream is provided to the synthesis reactor in the third stage so that the ratio of carbon monoxide and hydrogen in the synthesis reactor is about 1:2.

13. The system of claim 12, wherein the hydrogen stream is obtained via the first stage.

14. The system of claim 11, wherein the second stage comprises a shift reactor, adjacent to and downstream from the photocatalytic dry methane reformer, the shift reactor configured to produce the hydrogen stream, wherein the hydrogen stream obtained in the second stage is provided to the synthesis reactor in the third stage so that the ratio of carbon monoxide and hydrogen in the synthesis reactor is about 1:2.

15. The system of claim 11, wherein the second stage comprises a hydrogen separation membrane, adjacent to and downstream from the photocatalytic dry methane reformer,-and- wherein the hydrogen separation membrane is configured to produce the hydrogen stream, and wherein the hydrogen stream obtained in the second stage is provided to the synthesis reactor in the third stage so that the ratio of carbon monoxide and hydrogen in the synthesis reactor is about 1:2.

16. A method for transforming a methane feedstock into syngas, comprising:
providing the methane feedstock to a first stage comprising a photocatalytic steam methane reformer to obtain at least a carbon dioxide stream and a hydrogen stream; and
providing the carbon dioxide stream to a second stage comprising a photocatalytic dry methane reformer to produce the syngas.

17. The method of claim 16, wherein, in the first stage, the methane feedstock is provided to the photocatalytic steam methane reformer to form a first reaction product stream comprising hydrogen and carbon monoxide, followed by providing the first reaction product stream and water to a water-gas shift reactor to form a water-gas shift stream comprising hydrogen and carbon dioxide.

18. The method of claim 17, the method further comprising:
in the photocatalytic steam methane reformer, distributing the methane feedstock into at least one reactor cell disposed within a photocatalytic steam methane reformer housing, wherein each of the at least one reactor cell comprises an optically transparent enclosure and a first plasmonic photocatalyst on a first catalyst support disposed within the optically transparent enclosure;
illuminating, via at least one light source, the first plasmonic photocatalyst on the first catalyst support of each of the at least one reactor cell to cause the at least one reactor cell to transform the methane feedstock into the first reaction product stream comprising hydrogen and carbon monoxide; and
accumulating the first reaction product stream from the at least one reactor cell.

19. The method of claim 17, further comprising providing the water-gas shift stream comprising hydrogen and carbon dioxide to a separation unit to obtain the carbon dioxide stream and the hydrogen stream.

20. The method of claim 18, further comprising:
in the photocatalytic dry methane reformer, distributing the carbon dioxide stream and a second methane feedstock into at least one second reactor cell disposed within a photocatalytic dry methane reformer housing, wherein each of the at least one second reactor cell comprises an optically transparent enclosure and a second plasmonic photocatalyst on a second catalyst support disposed within the optically transparent enclosure;
illuminating, via at least one light source, the second plasmonic photocatalyst on the second catalyst support of each of the at least one second reactor cell to cause the at least one second reactor cell to transform the carbon dioxide and methane into the syngas; and
accumulating the syngas from the at least one second reactor cell.

21. A method for preparing methanol or dimethyl ether from a methane feedstock, comprising:
providing the methane feedstock to a first stage comprising a photocatalytic steam methane reformer to obtain at least a carbon dioxide stream and a hydrogen stream;
providing the carbon dioxide stream to a second stage comprising a photocatalytic dry methane reformer to produce the syngas; and
providing the syngas to a third stage comprising a synthesis reactor to obtain methanol or dimethyl ether.

22. The method of claim 21, further comprising providing a hydrogen stream to the synthesis rector in the third stage so that the ratio of carbon monoxide and hydrogen in the reactor is about 1:2.

23. The system of claim 3, wherein the photocatalytic steam methane reformer comprises:
a housing;
at least one reactor cell disposed within an interior of the housing, the at least one reactor cell comprising an enclosure and the first plasmonic photocatalyst on a first catalyst support disposed within the enclosure, wherein the enclosure is optically transparent and comprises at least one input for the methane feedstock to enter the at least one reactor cell and at least one output for the first reaction product stream to exit the at least one reactor cell; and
at least one light source, wherein, upon application of the at least one light source, the reactor cell is configured to form the first reaction product stream from the methane feedstock.

24. The system of claim 23, wherein the photocatalytic dry methane reformer comprises:
a second housing; and
at least one second reactor cell disposed within an interior of the second housing, the at least one second reactor cell comprising a second enclosure and a second plasmonic photocatalyst on a second catalyst support disposed within the second enclosure, wherein the second enclosure is optically transparent and comprises one or more inputs for the second methane feedstock and the carbon dioxide stream to enter the at least one second reactor cell and at least one output for the syngas to exit the at least one second reactor cell; and
at least one second light source, wherein, upon application of the at least one second light source, the second reactor cell is configured to form the syngas from the second methane feedstock and the carbon dioxide stream.
